(19) United States
(12) Patent Application Publication
(10) Pub. No.: US 2019/0331622 A1
(43) Pub. Date: Oct. 31, 2019

(54) Soil Moisture Monitoring System

(71) Applicant: China Agricultural University, Beijing (CN)

(72) Inventors: Qinglan Shi, Beijing (CN); Yili Ling, Beijing (CN); Yujiao Shi, Beijing (CN); Xiaochen Liu, Beijing (CN)

(21) Appl. No.: 16/039,990

(22) Filed: Jul. 19, 2018

(30) Foreign Application Priority Data
Apr. 28, 2018 (CN) 201810402282.6
Apr. 28, 2018 (CN) 201810403883.9

(51) Int. Cl. G01N 27/04

(52) U.S. Cl. CPC G01N 27/048; G01N 33/246

(57) ABSTRACT

The disclosure discloses a soil moisture monitoring system. The soil moisture monitoring system comprises a high-frequency signal source, for providing a soil moisture detection high-frequency signal; a sensing unit, comprising multiple nodes of sensing components arranged at intervals and in a layered manner and for sensing moisture of soil profiles at different depths under the action of the high-frequency signal source; a signal detection circuit, connected with the sensing unit and for generating a first voltage signal and a second voltage signal separately under the action of the high-frequency signal source; and a time division multiplexing switching unit, arranged between the sensing unit and the signal detection circuit and for conducting the sensing component in each layer and the signal detection circuit in a time division manner.

### CROSS REFERENCE TO RELATED APPLICATIONS

This application is based upon and claims priority to Chinese Patent Application No. 201810403883.9, filed on Apr. 28, 2018, and to Chinese Patent Application No. 201810402282.6, filed 28 Apr. 2018, titled “Soil Moisture Monitoring System”, and the entire contents of which are incorporated herein by reference.

### TECHNICAL FIELD

The disclosure relates to the technical field of soil moisture detection, and particularly to a soil moisture monitoring system.

### BACKGROUND

The soil moisture monitoring technology is widely applied to important monitoring projects such as agriculture, hydraulic engineering, weather, forestry and ecology; meanwhile, the soil moisture is also a significant constituent of soil fertility and an important influence factor of plant growth and development. The dynamic change of the soil moisture directly affects crop development evolution and crop yield. Therefore, to monitor the vertical distribution of the soil moisture in real time is of great importance to search on water demand rule of crop roots and formulation of reasonable irrigation strategies.

The traditional soil moisture detection method is generally to perform single-point detection on the soil profile, but the method for single-point detection of soil moisture is to directly read moisture data of the detection point by utilizing a handheld digital acquisition instrument. In order to monitor the condition of the water need of the plant roots, monitoring of the soil moisture at different depths is required, and the method is generally to integrate multiple independent analog sensing components and multiple independent detection circuits in a detecting tube. Due to the performance inconsistency of electronic components of the multiple independent detection circuits integrated in the detecting tube, consistent signal output of each independent analog sensing component installed in the detecting tube is difficult to guarantee, and reduction of the signal detection precision is accordingly caused. If each analog sensing component is enabled to output a consistent signal, manual work is required for debugging, and the debugging difficulty is relatively big. Obviously, more time and more labor force of debugging personnel are further consumed, and a relatively big error is caused.

### SUMMARY

Therefore, the technical problem to be solved by the embodiment is that due to integrated design of multiple independent detection circuits in the prior art, the performance inconsistency of the electronic components results in output inconsistency of detection signals, relatively big signal interference and reduction of the detection precision.

For this purpose, the embodiment of the disclosure provides the technical scheme as follows:

the embodiment of the disclosure provides a soil moisture monitoring system, comprising
a high-frequency signal source, for providing a soil moisture detection high-frequency signal;
a sensing unit, comprising multiple nodes of sensing components arranged at intervals and in a layered manner and for sensing moisture of soil profiles at different depths under the action of the high-frequency signal source;
a signal detection circuit, connected with the sensing unit and for generating a first voltage signal and a second voltage signal separately under the action of the high-frequency signal source; and
a time division multiplexing switching unit, arranged between the sensing unit and the signal detection circuit and for conducting the sensing component in each layer and the signal detection circuit in a time division manner.

Optionally, the soil moisture monitoring system further comprises

a first detecting tube, connected with the signal detection circuit and for performing voltage amplitude detection on the first voltage signal to obtain a first voltage parameter; and
a second detecting tube, connected with the signal detection circuit and for performing voltage amplitude detection on the second voltage signal to obtain a second voltage parameter.

Optionally, the soil moisture monitoring system further comprises a processing circuit, connected with the signal detection circuit and for calculating a difference value between the first voltage parameter and the second voltage parameter and performing A/D conversion on the difference value to obtain a moisture detection value.

Optionally, the soil moisture monitoring system further comprises a cloud platform, connected with the processing circuit through a wireless network.

Optionally, the time division multiplexing switching unit of the soil moisture monitoring system comprises a high-frequency switching switch which is connected with the signal detection circuit.

Optionally, the soil moisture monitoring system further comprises a solar cell panel, for providing a power source to the high-frequency signal source, the sensing unit, the signal detection circuit, the time division multiplexing switching unit, the processing circuit, the first detecting tube and the second detecting tube.

Optionally, the signal detection circuit of the soil moisture monitoring system further comprises a parallel high-frequency resonance circuit, connected with the high-frequency signal source and for generating the second voltage signal; and a series high-frequency resonance circuit, connected with the high-frequency signal source and for generating the first voltage signal.

Optionally, each node of the sensing components of the soil moisture monitoring system comprises a first metal ring and a second metal ring arranged at intervals.

Optionally, the first voltage signal and/or the second voltage signal are/is a high-frequency voltage signal.

Optionally, the first voltage parameter and/or the second voltage parameter are/is an analog voltage parameter.

The technical scheme of the embodiment has the advantages as follows:

the disclosure provides a soil moisture monitoring system which comprises a high-frequency signal source, for providing a soil moisture detection high-frequency signal; a sensing unit, comprising multiple nodes of sensing components arranged at intervals and in a layered manner and for sensing moisture of soil profiles at different depths under the action of the high-frequency signal source; a signal detection circuit, connected with the sensing unit and for generating a first voltage signal and a second voltage signal separately under the action of the high-frequency signal source; and a time division multiplexing switching unit, arranged between the sensing unit and the signal detection circuit and for conducting the sensing component in each layer and the signal detection circuit in a time division manner. Multiple nodes of the sensing components share the same signal detection circuit since the time division multiplexing switching unit conducts each node of the sensing components in the time division manner. Therefore, not only is consistency of output signals of the signal detection circuit guaranteed, but also the moisture detection precision is improved remarkably; and the circuit cost and the labor cost for circuit debugging are reduced.

### BRIEF DESCRIPTION OF THE DRAWINGS

In order to describe the technical schemes in the embodiments of the disclosure or the prior art more clearly, a simple introduction on the accompanying drawings which are needed in the specific embodiments or the prior art is given below. Apparently, the accompanying drawings in the description below are merely some of the embodiments of the disclosure, based on which other drawings may be obtained by those of ordinary skill in the art without any creative effort.

FIG. 1 is a structural block diagram of a soil moisture monitoring system in the embodiment 1 of the disclosure;

FIG. 2 is a structural block diagram of a sensing unit of the soil monitoring system in the embodiment 1 of the disclosure;

FIG. 3 is a high-frequency dual-resonance detection circuit in the embodiment 1 of the disclosure;

FIG. 4A is a relation curve between the soil moisture content and the first analog voltage signal of the soil moisture monitoring system in the embodiment 1 of the disclosure;

FIG. 4B is a graph of relation curve between the soil moisture content and the second analog voltage signal of the soil moisture monitoring system in the embodiment 1 of the disclosure;

FIG. 4C is a graph of relation curve between the soil moisture content and the detection voltage difference value of the soil moisture monitoring system in the embodiment 1 of the disclosure;

FIG. 5 is a schematic diagram of impedance of an RF lossless cable transmission line and a sensing unit of the soil moisture monitoring system in the embodiment 1 of the disclosure; and

FIG. 6 is an architecture diagram of the Internet of Things composed of the soil moisture monitoring system and a cloud platform in the embodiment 1 of the disclosure.

### DETAILED DESCRIPTION

A clear and complete description of the technical schemes in the embodiment of the present disclosure will be given below, in combination with the accompanying drawings. Apparently, the embodiments described below are a part, but not all, of the embodiments of the present disclosure. All of the other embodiments, obtained by those of ordinary skill in the art based on the embodiments of the present disclosure without any creative efforts, fall into the protection scope of the present disclosure.

In the description of the embodiments of the present disclosure, it should be noted that the orientation or position relationships indicated by the terms such as “center”, “up”, “down”, “left”, “right”, “vertical”, “horizontal”, “in” and “out” are the orientation or position relationships shown in the accompanying drawings, which are convenient for description of the embodiment of the disclosure and description simplification rather than indicating or hinting that the appointed devices or elements must have the specific orientation or be constructed and operated in the specific orientation. Accordingly, the orientation or position relationships indicated by the terms cannot be understood as the limitation to the disclosure. In addition, the terms, including, “first”, “second” and “third” are merely for describing the purpose and cannot be understood to indicate or hint the relative importance.

In the description of the embodiment of the disclosure, it should be noted that the terms such as “install”, “connect” and “connection” shall be understood broadly except for other specific regulation and limitation. For example, the connection can be fixed connection, detachable connection or integral connection; the connection can be mechanical connection or electric connection; and the connection may be direct connection, indirect connection through an intermediate medium, communication of the interiors of two elements, wireless connection or wired connection. For those of ordinary skill in the art, the specific meanings of the terms in the disclosure can be understood based on the specific condition.

In addition, the technical characteristics involved in different embodiments of the disclosure below can be combined mutually as long as no conflict exists among the technical characteristics.

### Embodiment 1

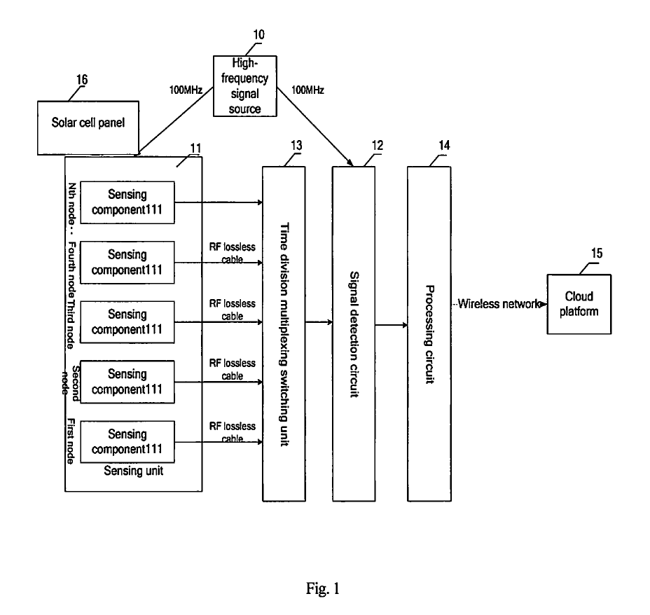

The embodiment of the disclosure provides a soil moisture monitoring system, which, as shown in FIG. 1, comprises a high-frequency signal source 10, a sensing unit 11, a signal detection circuit 12, a time division multiplexing switching unit 13, a processing circuit 14, a cloud platform 15 and a solar cell panel 16.

The soil moisture monitoring system in the embodiment further comprises a first detecting tube and a second detecting tube, which are separately connected with the signal detection circuit 12,

wherein, the high-frequency signal source 10 is used for providing a soil moisture detection high-frequency signal. The high-frequency signal source 10 herein can generate a 100 MHz high-frequency signal.

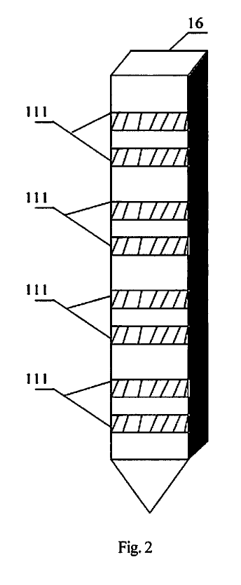

As shown in the FIG. 1 and FIG. 2, the sensing unit 11 comprises multiple nodes of sensing components 111 which are arranged at intervals and in a layered manner; multiple nodes of the sensing components 111 are installed on a detecting tube; and each node of the sensing components 111 further comprises a first metal ring and a second metal ring which are arranged at intervals. The detecting tube is made of an anti-corrosion and high-low temperature resistant insulating material and has the dielectric constant in the range of 3.0-3.2; and the first metal rings and the second metal rings can be made of stainless steel rings. Each node of the sensing components 111 of the sensing unit 11 detects the soil moisture of the respective soil profile at each depth. For example, the soil moisture monitoring system in the embodiment is used for detecting the soil moisture, and the soil depth at the moment is 80 cm; the sensing unit 11 comprises four nodes of the sensing components 111; and the first node of the sensing components 111 is used for detecting the soil moisture at the soil depth of 20 cm, the second node of the sensing component 111 is used for detecting the soil moisture at the soil depth of 40 cm, the third node of the sensing components 111 is used for detecting the soil moisture at the soil depth of 60 cm, and the fourth node of the sensing components 111 is used for detecting the soil moisture at the soil depth of 80 cm. Therefore, the sensing unit 11 in the embodiment can detect the soil moisture of the soil profiles at different depths. Of course, as other substitutable embodiments, the sensing unit 11 in the soil moisture monitoring system in the embodiment also can be used for detecting other different porous mediums. For example, the sensing unit 11 can detect wheat piled with a certain depth, the sensing unit 11 also can detect sand piled with a certain depth, and the sensing unit 11 also can detect rice piled with a certain depth. The soil moisture monitoring system in the embodiment is provided with multiple nodes of the sensing components 111 which can detect moisture values of the soil profiles at different depths simultaneously, so that the detection efficiency can be increased remarkably, and the detection time can be reduced. Specifically, each node of the sensing components 111 of the sensing unit 11 is used for enabling the formed capacitive reactance to connect with the signal detection circuit 12 through an RF lossless cable under the action of the high-frequency signal source 10; the sensing unit 11 senses the moisture of the soil profile at each depth.

The signal detection circuit 12, connected with the sensing unit 11, is used for generating a first voltage signal and a second voltage signal separately under the action of the high-frequency signal source 10; the first voltage signal is subjected to voltage amplitude detection through the first detecting tube to obtain a first voltage parameter and the second voltage signal is subjected to voltage amplitude detection through the second detecting tube to obtain a second voltage parameter. The first voltage signal and the second voltage signal herein can be a high-frequency voltage signal, the high-frequency voltage signal is a sinusoidal alternating current, namely the first voltage signal and the second voltage signal are high-frequency analog voltage signals, and therefore, the first voltage signal and the second voltage signal are required to be subjected to detection by the first detection tube and the second detection tube, respectively, and the first voltage parameter and the second voltage parameter obtained by detection are analog voltage parameters.

The processing circuit 14, connected with the signal detection circuit 12, is used for calculating a difference value between the first voltage parameter and the second voltage parameter and performing A/D conversion on the difference value to obtain a moisture detection value. The difference value ensures that the output always varies within a monotone interval when the soil moisture changes.

The signal detection circuit 12 comprises a parallel high-frequency resonance circuit and a series high-frequency resonance circuit which can compose a high-frequency dual-resonance detection circuit; the high-frequency dual-resonance detection circuit can ensure that the relationship between the difference value of the second voltage parameter and the first voltage parameter in the soil moisture dry-to-wet changing process and the soil moisture is a monotonically increased function relationship.

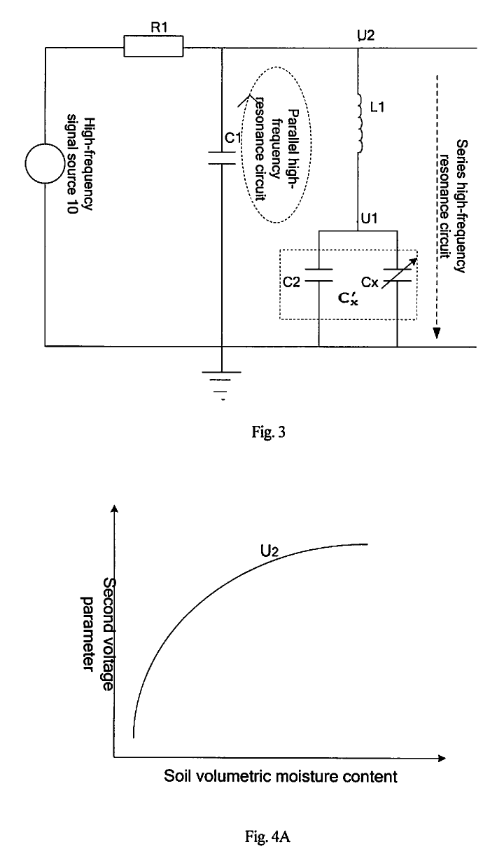

Specifically, the parallel high-frequency resonance circuit and the series high-frequency resonance circuit are separately connected with the high-frequency signal source 10. The parallel high-frequency resonance circuit is used for generating the second voltage signal under the action of the high-frequency signal source 10, and the series high-frequency resonance circuit is used for generating the first voltage signal under the action of the high-frequency signal source 10. To be specific, as shown in FIG. 3, one end of the high-frequency signal source 10 is separately connected with one end of a capacitor C1 and one end of an inductor L1 by a resistor R1; the other end of the inductor L1 is separately connected with one end of a capacitor C2 and one end of a capacitor Cx; the other end of the high-frequency signal source 10 is grounded with one end of the capacitor C1, the other end of the capacitor C2, and the other end of the capacitor Cx; the capacitor C2 and the capacitor Cx are connected in parallel; an equivalent of the capacitor C2 and the capacitor Cx connected in parallel is a capacitor Cx′; the capacitor Cx′ and the inductor L1 are connected in series. When the soil moisture content is relatively high, the inductor L1 and the capacitor Cx′ are inductive, and a resulting equivalent inductor, the capacitor C1 and the inductor L1 compose the parallel high-frequency resonance circuit under the action of the high-frequency signal source 10. When the soil moisture content is relatively low, the Cx is relatively low, and the high-frequency signal source 10, the inductor L1 and the capacitor Cx′ compose the series high-frequency resonance circuit through the resistor R1.

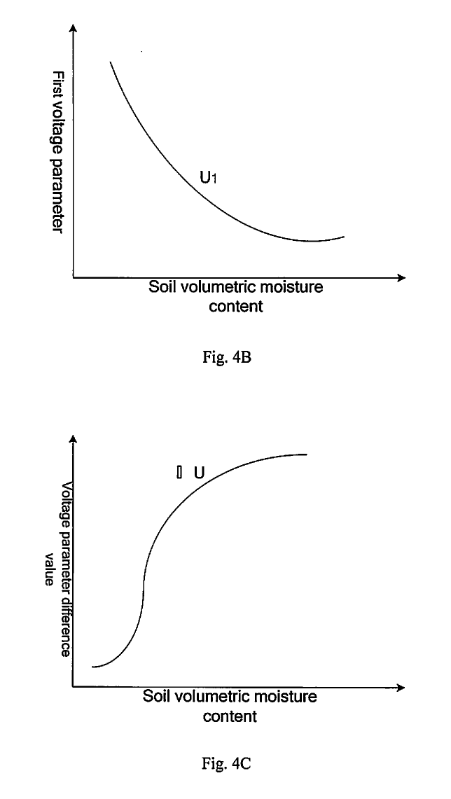

For example, when the soil moisture monitoring system in the embodiment is used for detecting the soil moisture, two ends of each sensing component 111 of the sensing unit 11 are in contact with the soil. At the moment, the sensing components 111 can constitute the equivalent capacitor Cx in the FIG. 3. The high-frequency double-resonance detection circuit composed of the signal detection circuit 12 has high sensitivity to the soil moisture; the second voltage signal is generated by the parallel high-frequency resonance circuit under the action of the high-frequency signal source 10; the first voltage signal is generated by the series high-frequency resonance circuit under the action of the high-frequency signal source 10. The second voltage signal is subjected to voltage amplitude detection by the second detecting tube to obtain a second voltage parameter U₂; and the first voltage signal is subjected to voltage amplitude detection by the first detecting tube to obtain a first voltage parameter U₁. When the sensing components 111 have no contact with the soil, the second voltage parameter U₂ is smaller than the first voltage parameter U₁, that is to say, U₂<U₁. When each sensing component 111 is in contact with dry soil, the sensing components 111 are connected in series and perform resonant oscillation in the presence of a 100 MHz high-frequency sinusoidal wave frequency signal, that is to say, when each node of the sensing components 111 of the sensing unit 11 is inserted into the dry soil (the moisture is 0%, and the moisture is in a drying state), the second voltage parameter U₂ is slightly smaller than the first voltage parameter U₁, that is to say, U₂≈U₁. When the soil moisture changes from dry to wet, that is to say, as shown in the FIG. 4A-4B, the capacitor Cx becomes bigger, the amplitude of the U₂ is increased gradually, and the amplitude of the U₁ is decreased gradually. When the moisture is increased continuously until the moisture is saturated, the amplitude of the U₂ approaches steady, and the moisture voltage is increased slowly after the moisture is saturated. In the FIG. 4A and the FIG. 4B, the specific change process is that the relationships between the U₂ and the soil volumetric moisture content as well as between the U₁ and the oil volumetric moisture content are monotonic increase and monotonic decrease, so that the U₂-U₁ and the soil volumetric moisture content are in the monotonic increase relationship, Uₒᵤₜ=U₂−U₁=ΔU; Uₒᵤₜ changes along with the change of the soil volumetric moisture content, is related to a soil dielectric constant, and is accordingly related to the soil moisture content. Specifically, the soil moisture value at the position can be detected through contact between two ends of each node of the sensing components 111 of the sensing unit 11 and the soil, the impedance Zᵢ at the joint of each node of the sensing components 111 is equal to

$$- j \frac{{z}_{c}}{\sqrt{ɛ}} ctg \frac{2 π \sqrt{ɛ}}{{λ}_{0}} 1 , \qquad ( 1 )$$

wherein Z_{c} is the characteristic impedance of a probe in air, l is the length of the probe, λ₀ is the wavelength of the testing sinusoidal wave signal in air, ε is the dielectric constant of the soil around the probe, and j is an expression factor of the imaginary part. From the formula (1), the metal sensing rings of each node of the sensing components 111 take on capacitive impedance in a medium, and the impedance changes along with the change of the soil volumetric moisture content (that is to say, changes along with the change of the ε). As a result, Uₒᵤₜ changes along with the change of the soil volumetric moisture content, that is to say, the Uₒᵤₜ is related to the soil dielectric content and is accordingly related to the soil moisture content. The high-frequency double-resonance detection circuit guarantees that the relationship between |ΔU|=|U₂−U₁| and the soil moisture in the soil moisture dry-to-wet changing process is a monotonically increased function relationship, so that the processing circuit 14 can judge the moisture content value of the soil moisture by calculating the amplitude of the difference value AU of the second voltage parameter and the first voltage parameter, as shown in the FIG. 4C.

The time division multiplexing switching unit 13, arranged between the sensing unit 11 and the signal detection circuit 12, is used for conducting the sensing components 111 in each layer and the signal detection circuit 12 in the time division manner and comprises a high-frequency switching switch which is connected with the signal detection circuit 12.

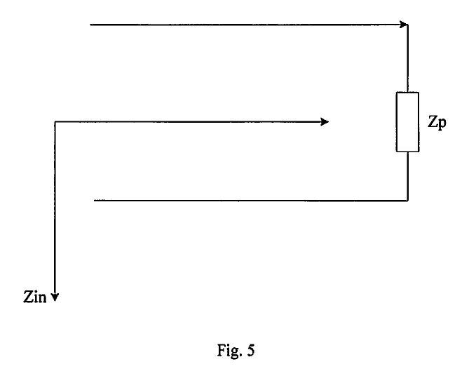

The time division multiplexing switching unit 13 herein is similar to a single-pole multi-throw switch in an electric component and can switch the circuit at any time. Here, the time division multiplexing switching unit 13 is connected with each node of the sensing components 111 of the sensing unit 11 by the RF lossless cable; if there are four nodes of the sensing components 111 of the sensing unit 11 in the embodiment, each RF lossless cable is preferably 99 cm in length and has relatively small impedance. The signal attenuation is relatively low; the traditional cable is generally a coaxial cable which may add one additional impedance and one inductive impedance to each node of the sensing components 111 and will change the circuit parameters, and reduce the sensitivity of the sensing components 111; in addition, the inconsistent length of the traditional coaxial cable may also result in phase disorder of signals, thereby causing signal output disorder. However, a large number of experiments prove that the RF lossless cable in the embodiment has relatively low impedance and thus a signal transmission effect is optimal. For example, the RF lossless cable is adopted to guarantee no attenuation loss of the high-frequency signal, and the length of the RF lossless cable is a half wavelength of the high-frequency signal. If the frequency of the high-frequency signal is 100 MHz,

$$1 = \frac{λ}{2 \sqrt{ɛ}} = \frac{3 \times {10}^{8} / 100 \times {10}^{6}}{2 \sqrt{2.3}} = 0.99 , \qquad ( 2 )$$

wherein ε is the dielectric constant of a polytetrafluoroethylene insulating layer of the RF lossless cable and is about 2.3. What is described below is about hypothesis of the length of the RF lossless cable; as shown in FIG. 5, the end from the signal detection circuit 12 serves as a transmission line inlet, and a terminal is an input impedance Zᵢₙ of a transmission line, connected to Zₚ of the sensing component 111 for soil moisture detection;

$${Z}_{i n} = {Z}_{c} \cdot \frac{{Z}_{p} + {jZ}_{c} tgax}{{Z}_{c} + {jZ}_{p} tgax} , \qquad ( 3 )$$

wherein Z_{c} is the characteristic impedance of the transmission line, Zₚ is the load impedance of the terminal of the transmission line, that is, the impedance of the probe, x is the distance to the terminal;

$$ax = ω \frac{x}{C} = 2 π \cdot \frac{x}{λ} , \qquad ( 4 )$$

wherein λ is the wavelength of the 100 MHz high-frequency signal, so that when

$$x = \frac{k λ}{2} ( k = 0 , 1 , 2 , 3 … ) ,$$

$$tg \frac{2 π}{λ} \cdot x = 0 ,$$

and Zᵢₙ=Zₚ, and accordingly

$${Z}_{i n} = {Z}_{c} \cdot \frac{{Z}_{p} + {jZ}_{c} tg \frac{2 π}{λ} x}{{Z}_{c} + {jZ}_{p} tg \frac{2 π}{λ} x} . \qquad ( 5 )$$

Therefore, when

$$x = k \frac{λ}{2} ( k = 0 , 1 , 2 … ) ,$$

$$tg \frac{2 π}{λ} ≅ 0.$$

At the moment, as shown in the FIG. 5, Zᵢ=Zₚ, this means that the equivalent output is directly connected with the sensing components 111, the RF lossless cable is a high-performance decimeter wave cable for preventing length change. As a result, the length of the RF lossless cable obtained through the derivation process abovementioned when being integral multiple of the high-frequency electromagnetic wave can cancel the impedance value of the transmission line per se, and what is connected to the signal detection circuit 12 is purely the capacitive impedance of the metal sensing ring of each sensing component 111.

As the time division multiplexing switching unit 13 is directly connected with the signal detection circuit 12, the high-frequency switching switch in the time division multiplexing switching unit 13 conducts the signal detection circuit 12 in the time division manner to enhance the consistency of signal output without performing integrated design on multiple independent detection circuits for detecting the soil moisture at multiple depths in the prior art; and the integrated design of the multiple independent detection circuits results in relatively poor consistency of the signal output. For example, sensors of three depths are subjected to consistency calibration in the same soil sample. Three 8 Kg soil moisture samples below, used for verifying the consistency of the three sensors, are made by using a drying method: a 4.75% soil moisture sample, a 17.79% soil moisture sample and a 40.1% soil moisture sample, respectively.

TABLE 1

| Weight moisture | | | | |
|---|---|---|---|---|
| content by using | H1 output | H2 output | H3 output | Average |
| drying method | voltage | voltage | voltage | output |
| (g/g) | (V) | (V) | (V) | (V) |
| 4.75 | 0.573 | 0.564 | 0.581 | 0.572666667 |
| 17.79 | 1.06 | 1.12 | 1.01 | 1.063333333 |
| 40.1 | 1.82 | 1.83 | 1.82 | 1.823333333 |
| RMSE | 0.03332 | 1.2808 | 1.2477 | |

The output consistency of voltages at three depths is evaluated by adopting a root-mean-square error

$$RMSE = \sqrt{\frac{1}{n} {\sum }_{i = 0}^{n} {( {x}_{i} - \overline{x} )}^{2}} ,$$

wherein n is the number of measuring sample points used for regression analysis and is equal to 3 here, xᵢ is the output value measured at the ith sample point, and x̅ is the average of the output values of all the sample points. The root-mean-square errors of the three depth measurement values, calculated by using the measurement values in the Table 2 and the formulas are approximately 0.03332, 1.2808 and 1.2477, respectively. This shows that the consistency of the three depth working points is relatively poor.

The embodiment of the disclosure aims at overcoming mutual interference caused by integration of multiple independent detection circuits, performance inconsistency of electronic components of the multiple detection circuits and difficulty in guaranteeing consistency of signal output of multiple circuit boards in the prior art. In the embodiment, the time division multiplexing switching unit 13 is adopted to switch the soil at different position depths to a corresponding node of the sensing components 111 and is connected with the same signal detection circuit 12 to detect the moisture value at each depth by turns. Supposing that N depths are required to be detected, the detection duration is divided into N time intervals, the time division multiplexing switching unit 13 is adopted for gating of each detection channel by turns, and the sensing components 111 corresponding to the channel are connected with the signal detection circuit 12.

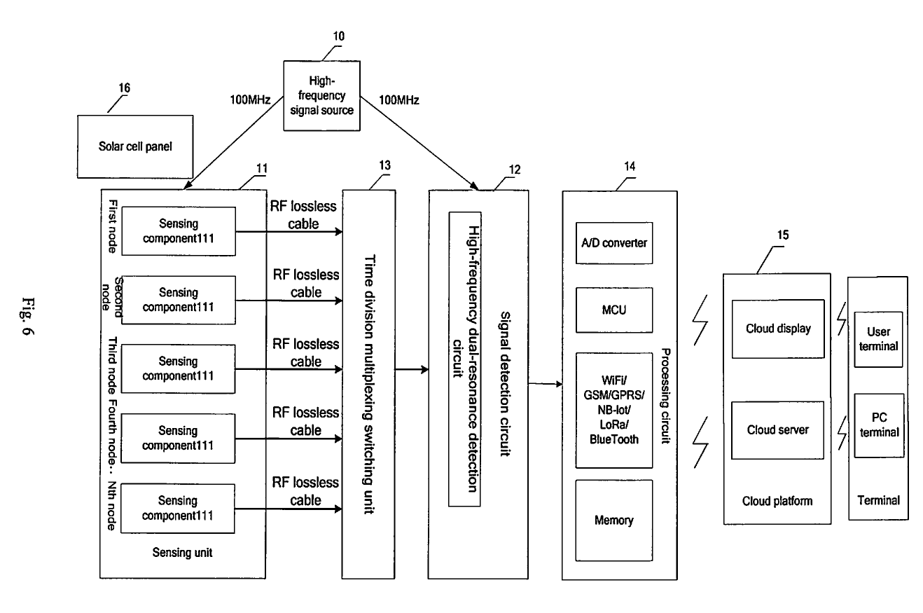

The cloud platform 15, connected with the processing circuit 14 through a wireless network, is a cloud server platform; and the wireless network is one or more of Wifi, GSM, GPRS, NB-lot, LoRa or Bluetooth, and has a long transmission distance and a good transmission effect. The processing circuit 14 obtains the moisture detection values and transmits the moisture detection values to the cloud platform 15 through the wireless network to realize a real Internet of Things; the processing circuits 14 is directly connected with a user terminal, the cloud platform 15, the Internet of Things and the big data without any additional Ad-Hoc Network so as to further realize interconnection and mutual communication of massive users and the soil moisture monitoring system; and users can obtain soil moisture content information at each place everywhere and anytime by virtue of any mobile terminal. Through the system architecture diagram of the Internet of Things and the soil moisture monitoring system in the embodiment of the disclosure, as shown in the FIG. 6, low-cost coverage of big-area Internet of Things can be realized really at a long distance, low power consumption and low operation and maintenance costs; meanwhile, the spread-spectrum ultra-long distance LoRa and NB-lot technology also can be used for supporting the massive users; a hybrid developed APP (Hybrid App) technology is adopted, that is, a light-weight browser is embedded in one APP, and partial functions are developed by adopting HTML5. Dynamic update can be carried out without updating the APP and the system can be run at the APP of Android or iOS. Therefore, not only can the development resources be saved, but also users are enabled to have good user experience. The cloud platform 15 can display the uploaded information of the soil moisture monitoring system, query historical data, send query and work instructions, monitor the state and send an alarm for data abnormality.

The soil moisture monitoring system in the embodiment further comprises the solar cell panel 16, installed on the detecting tube of the sensing unit 11 and for providing a power source to the high-frequency signal source 10, the sensing unit 11, the signal detection circuit 12, the time division multiplexing switching unit 13, the first detecting tube, the second detecting tube and the processing circuit 14. The solar cell panel 16 has the photovoltaic nominal voltage of 6V and can guarantee that the system can work outdoors continuously without any people on duty. The system can acquire the moisture content of the soil volume every an hour, is energy-saving and environment-friendly, and can provide the power source to the high-frequency signal source 10, the sensing unit 11, the signal detection circuit 12, the time division multiplexing switching unit 13, the first detecting tube, the second detecting tube and the processing circuit 14 continuously.

The soil moisture monitoring system in the embodiment not only can be applied to the soil moisture detection, but also can perform moisture detection on grain, wheat or sand piled with a certain depth and also can realize integrated, small-sized and systematic design of analog sensing, data acquisition, wireless communication, cloud server and user terminal. Users can directly access data provided by the Internet of Things and the cloud platform 15 and can query the detection data, the work state, the historical data line chart and scatter diagram and the like of the soil moisture monitoring system through the intelligent terminal APP. What is most important of the soil moisture monitoring system in the embodiment is that each node of the sensing components 111 in each channel after being conducted in a time division manner through the time division multiplexing switching unit 13 is directly connected with the same signal detection circuit 12, the first detecting tube and the second detecting tube perform the amplitude detection under the action of the high-frequency signal source 10 to obtain the first voltage parameter and the second voltage parameter, the first voltage parameter and the second voltage parameters are directly converted into digital signals through the processing circuit 14 rather than differential amplification of an operational amplifier, and a single-chip microcomputer calculates out a moisture content through a calibration equation, or the digital signals are stored by the single-chip microcomputer and are sent to the cloud platform 15 through GSM/GPRS/NB-lot wirelessly. The moisture content is calculated through the calibration equation stored in the cloud platform 15, and the consistency of the signals output from multiple channels is relatively good and the error is reduced; and relatively poor consistency of the signal output caused by the traditional integrated design of multiple independent detection circuits is avoided.

The sensing unit 11 of the soil moisture monitoring system provided by the embodiment of the disclosure may further comprise multiple temperature-sensitive resistors, for detecting the soil temperature. As shown in the FIG. 1 and FIG. 6, the sensing unit 11 is connected with the time division multiplexing switching unit 13 through the RF lossless cables; the time division multiplexing switching unit 13 conducts each high-frequency switching switch of the time division multiplexing switching unit 13 in the time division manner to enable each node of the sensing components 111 to be directly connected with the same signal detection circuit 12. The soil moisture values and temperature values at different depths are detected by turns so as to enable the signal detection circuit 12 to finally output stable and consistent detection signals, thereby increasing the soil moisture detection precision. Supposing that N depths are required to be detected, the detection duration is divided into N time intervals, and the time division multiplexing switching unit 13 is adopted for gating of each detection channel by turns, and the sensing components 111 corresponding to the channel are connected with the signal detection circuit 12. The problems of mutual signal interference caused by integration of multiple independent detection circuits and performance inconsistency of electronic components of the multiple independent detection circuits in the prior art can be overcome; and consistency in signal output of multiple circuit boards can be guaranteed.

Furthermore, the soil moisture monitoring system in the embodiment of the disclosure, due to adoption of the cloud platform 15, can realize terminal-cloud integrated monitoring of the soil moisture and temperature at multiple depths. As shown in the FIG. 6, the processing circuit 14 obtains the moisture detection values and transmits the moisture detection values and the temperature detection values to the cloud platform 15 through the wireless network to realize a real Internet of Things; the processing circuit 14 is directly connected with the cloud platform 15, the Internet of Things and the big data without any additional Ad-Hoc Network so as to further realize interconnection and mutual communication of massive users and the soil moisture monitoring system; and users can obtain soil moisture content information at each place everywhere and anytime by virtue of any mobile terminal. Low-cost coverage of the big-area Internet of Things can be realized really at a long distance, low power consumption and low operation and maintenance cost.

Obviously, the embodiment is merely an example to illustrate the disclosure clearly, rather than limiting the embodiment. It should be understood by those of ordinary skill in the art that changes or modifications in other different forms may still be made based on the disclosure. All the embodiments here cannot be illustrated. These apparent changes or modifications still fall in the protection scope of the disclosure.

What is claimed is:

1. A soil moisture monitoring system, comprising
a high-frequency signal source, for providing a soil moisture detection high-frequency signal;
a sensing unit, comprising multiple nodes of sensing components arranged at intervals and in a layered manner and for sensing moisture of soil profiles at different depths under the action of the high-frequency signal source;
a signal detection circuit, connected with the sensing unit and for generating a first voltage signal and a second voltage signal separately under the action of the high-frequency signal source; and
a time division multiplexing switching unit, arranged between the sensing unit and the signal detection circuit and for conducting the sensing component in each layer and the signal detection circuit in a time division manner.

2. The soil moisture monitoring system of claim 1, further comprising
a first detecting tube, connected with the signal detection circuit and for performing voltage amplitude detection on the first voltage signal to obtain a first voltage parameter; and
a second detecting tube, connected with the signal detection circuit and for performing voltage amplitude detection on the second voltage signal to obtain a second voltage parameter.

3. The soil moisture monitoring system of claim 2, further comprising
a processing circuit, connected with the signal detection circuit and for calculating a difference value between the first voltage parameter and the second voltage parameter and performing A/D conversion on the difference value to obtain a moisture detection value.

4. The soil moisture monitoring system of claim 3, further comprising a cloud platform, connected with the processing circuit through a wireless network.

5. The soil moisture monitoring system of claim 1, wherein, the time division multiplexing switching unit comprises a high-frequency switching switch which is connected with the signal detection circuit.

6. The soil moisture monitoring system of claim 1, further comprising a solar cell panel, for providing a power source to the high-frequency signal source, the sensing unit, the signal detection circuit, the time division multiplexing switching unit, the processing circuit, the first detecting tube and the second detecting tube.

7. The soil moisture monitoring system of claim 1, wherein, the signal detection circuit further comprises a parallel high-frequency resonance circuit, connected with the high-frequency signal source and for generating the second voltage signal; and
a series high-frequency resonance circuit, connected with the high-frequency signal source and for generating the first voltage signal.

8. The soil moisture monitoring system of claim 1, wherein, each node of the sensing components comprises a first metal ring and a second metal ring arranged at intervals.

9. The soil moisture monitoring system of claim 1, wherein, the first voltage signal and/or the second voltage signal are/is a high-frequency voltage signal.

10. The soil moisture monitoring system of claim 2, wherein, the first voltage signal and/or the second voltage signal are/is a high-frequency voltage signal.

11. The soil moisture monitoring system of claim 3, wherein, the first voltage signal and/or the second voltage signal are/is a high-frequency voltage signal.

12. The soil moisture monitoring system of claim 4, wherein, the first voltage signal and/or the second voltage signal are/is a high-frequency voltage signal.

13. The soil moisture monitoring system of claim 5, wherein, the first voltage signal and/or the second voltage signal are/is a high-frequency voltage signal.

14. The soil moisture monitoring system of claim 6, wherein, the first voltage signal and/or the second voltage signal are/is a high-frequency voltage signal.

15. The soil moisture monitoring system of claim 7, wherein, the first voltage signal and/or the second voltage signal are/is a high-frequency voltage signal.

16. The soil moisture monitoring system of claim 8, wherein, the first voltage signal and/or the second voltage signal are/is a high-frequency voltage signal.

17. The soil moisture monitoring system of claim 3, wherein, the first voltage parameter and/or the second voltage parameter are/is an analog voltage parameter.
